(19) United States
(12) Patent Application Publication
(10) Pub. No.: US 2006/0276787 A1
(43) Pub. Date: Dec. 7, 2006

(54) Pedicle screw, cervical screw and rod

(75) Inventors: Rafail Zubok, Midland Park, NJ (US); Mikhail Kvitnitsky, Clifton, NJ (US)

Correspondence Address: KAPLAN GILMAN GIBSON & DERNIER L.L.P., 900 ROUTE 9 NORTH, WOODBRIDGE, NJ, 07095, US

(73) Assignee: ACCIN Corporation

(21) Appl. No.: 11/137,963

(22) Filed: May 26, 2005

(51) Int. Cl. A61F 2/30

(52) U.S. Cl. 606/61

(57) ABSTRACT

A rod for interconnecting at least two pedicle screws implanted in adjacent vertebrae of a spine and/or the rod itself includes a metal or metal alloy of substantial nickel content; and an outer layer of ceramic coating the metal such that exposure of the nickel to a patient is inhibited.

### BACKGROUND OF THE INVENTION

This invention relates generally to an apparatus for immobilization of the spine, and more particularly, to an apparatus for posterior internal fixation of the spine as well as to a method of therapy which utilizes the device. For example, the present invention relates to pedicle screws and rods for fixing vertebrae in a spine.

Various methods of spinal immobilization have been known and used during this century in the treatment of spinal instability and displacement. One treatment for spinal stabilization is immobilization of the joint by surgical fusion, or arthrodesis. This method has been known since its development in 1911 by Hibbs and Albee. However, in many cases, and in particular, in cases involving fusion across the lumbosacral articulation and when there are many levels involved, pseudoarthrosis is a problem. It was discovered that immediate immobilization was necessary in order to allow a bony union to form.

Internal fixation refers to therapeutic methods of stabilization which are wholly internal to the patient and include commonly known devices such as bone plates and pins. External fixation in contrast involves at least some portion of the stabilization device which is external to the patient's body. Internal fixation is advantageous since the patient is allowed greater freedom with the elimination of the external portion of the device and the possibility of infections, such as pin tract infection, is reduced.

Some of the indications treated by internal fixation of the spine include vertebral displacement and management such as kyphosis, spondylolisthesis and rotation; segmental instability, such as disc degeneration and fracture caused by disease and trauma and congenital defects; and tumor diseases.

A common problem with spinal fixation is the question of how to secure the fixation device to the spine without damaging the spinal cord. The pedicles are a favored area of attachment since they offer an area that is strong enough to hold the fixation device even when the patient suffers from osteoporosis. Since the middle 1950's, methods of fixation have utilized the pedicles. In early methods, screws extended through the facets into the pedicles. Posterior methods of fixation have been developed which utilize wires that extend through the spinal canal and hold a rod against the lamina (such as the Luque system).

A conventional system for interconnecting two vertebrae of a spine includes a pair of pedicle screws, a rod spanning the screws and connecting means for fixing the rod to the pedicle screws. Connecting means have been developed that permit the pedicle screw to take various articulation angles with respect to the rod so that desirable positions may be obtained. The problem with conventional pedicle screws and rods is that they are relatively large, resulting in reduced degrees of freedom in terms of articulation, reduced purchase strength of the screw to the bone, reduced degrees of freedom in terms of positioning the pedicle screws, rod, and connecting means to a particular patient. This is so because of design balances that have been made as between material strength and bio-acceptability.

Titanium alloys have typically been used as bio-acceptable materials for forming the pedicle screws and rods. Titanium, however, cannot withstand high bending stresses as compared with other metals, such as steel alloys (e.g., stainless steel) and cobalt chromium. Given the relatively poor mechanical strength properties of titanium, pedicle screws and rods formed thereof are dimensioned relatively large in order to meet design margins.

Accordingly, there is a need in the art for a new system for immobilization of the spine, which may employ pedicle screws and rods that exhibit improved mechanical performance as compared with the conventional approaches.

### SUMMARY OF THE INVENTION

In accordance with one or more embodiments of the present invention, a rod for interconnecting at least two pedicle screws implanted in adjacent vertebrae of a spine includes: an elongate shaft formed of a metal or metal alloy of substantial nickel content (higher than about 0.3%); and an outer layer of ceramic coating the shaft such that exposure of the nickel to a patient is inhibited. The shaft may be formed of at least one of a cobalt chromium alloy and a steel alloy. The outer layer may be formed of titanium nitrite. The shaft may be formed of stainless steel.

In accordance with one or more further embodiments of the present invention, a pedicle screw for implantation into spinal vertebrae includes: a metal or metal alloy of substantial nickel content; and an outer layer of ceramic coating the metal such that exposure of the nickel to a patient is inhibited. The metal or metal alloy is formed of at least one of a cobalt chromium alloy and a steel. The metal may be formed of stainless steel. The outer layer may be formed of titanium nitrite.

Other aspects, features, and advantages of the present invention will be apparent to one skilled in the art from the description herein taken in conjunction with the accompanying drawings.

### DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a perspective view of a system for immobilization of the spine in accordance with one or more embodiments of the present invention;

FIGS. 2A-2B are side and cross-sectional views, respectively, of an interconnecting rod that may be employed in the system for immobilization of the spine of FIG. 1 in accordance with one or more embodiments of the present invention;

FIG. 3 is a side view of a pedicle screw and rod assembly illustrating bending stress and flexion that may be impressed on an interconnecting rod that may be employed in the system for immobilization of the spine of FIG. 1;

FIG. 4 is a side view of a pedicle screw that may be employed in the system for immobilization of the spine of FIG. 1 in accordance with one or more embodiments of the present invention;

FIG. 5 is a cross sectional view of a threaded portion of a pedicle screw that may be employed in the system for immobilization of the spine of FIG. 1 and that exhibits certain thread purchase properties in accordance with one or more embodiments of the present invention; and

FIG. 6 is a cross sectional view of a pedicle screw and tulip that may be employed in the system for immobilization of the spine of FIG. 1 and that exhibit certain articulation angle properties in accordance with one or more embodiments of the present invention.

### DETAILED DESCRIPTION OF THE PREFERRED EMBODIMENTS

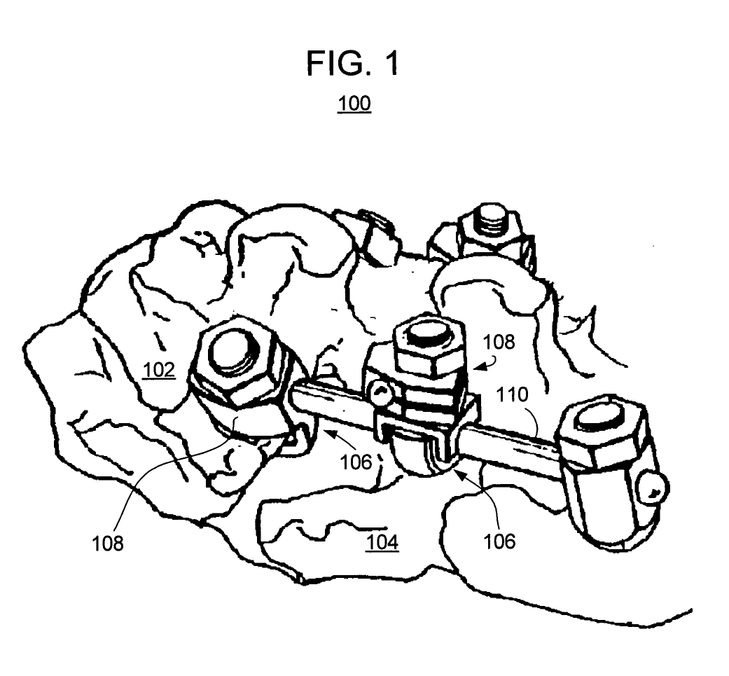

With reference to the drawings wherein like numerals indicate like elements there is shown in FIG. 1 an anchoring system 100 for internal fixation of respective vertebral bones 102, 104 of a patient. The system 100 includes a plurality of pedicle screws 106 and anchor seats (or tulips) 108 that cooperate to fix a portion of a rod 110 to a bone. Although in some embodiments of the invention the specific design details of the pedicle screws 106 and anchor seats 108 are not of significant concern, it is noted here that the anchor seats 104 may include a socket and a locking element, and the pedicle screw 106 may include a head. The socket is preferably sized and shaped to receive a corresponding contour of the head of the pedicle screw 106 to permit articulation of the anchor seat 108 relative to the pedicle screw 106. The locking element of the anchor seat 108 fixes the relative positions of the pedicle screw 106 and the rod 110 after all the components are in a desired position.

The operative procedure for installation of the system 100 preferably includes inserting the pedicle screws 106 into the bores of the bones 102, 104, engaging the rod 110 with the anchor seats 108, articulating the anchor seats 108 into a desired position with respect to the pedicle screw 106 and the rod 110, and tightening the locking element of the anchor seat 108 to fix the rod 110 with respect to the anchor seat 108 and fix the rod 110 with respect to the pedicle screw 106. Thus, all components of the system 100 achieve a rigid and fixed orientation with respect to one another.

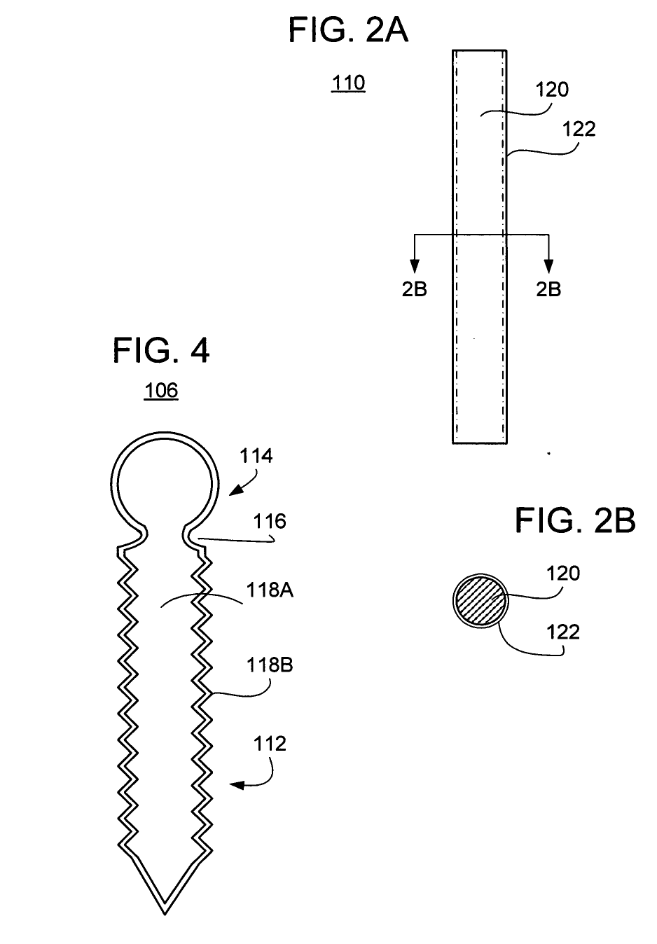

Reference is now made to FIG. 2A, which is a side view and FIG. 2B, which is a cross-sectional view along line 2B-2B, respectively, of the interconnecting rod 110 of the system 100 for immobilization of the spine. The rod 110 is of generally elongate construction and generally circular cross section. It is understood, however, that other cross-sectional configurations, such as oval, square, rectangular, etc. are within the scope of the various embodiments of the invention. An elongate shaft portion 120 of the rod 110 is preferably formed of a first metal or metal alloy. Notably, the first metal or metal alloy may have substantial nickel content, such as greater than about 0.3%. For example, the first metal or metal alloy may be a cobalt chromium alloy, stainless steel, etc. The rod also preferably includes an outer layer 122 of ceramic coating the shaft 120 such that exposure of nickel to the patient, or migration of the nickel into the patient, is inhibited. For example, the outer ceramic layer 122 may be titanium nitrite. The thickness of the ceramic outer layer 122 is preferably in the range of about 0.25 to 12 microns. The ceramic outer layer 122 is preferably deposited onto the shaft 120 using the Physical Vapor Deposition (PVD) vacuum system or any other of the known and suitable approaches for bonding materials together.

In patients with nickel sensitivity, a relatively high level of nickel (higher than about 0.3%) in an implanted device may be intolerable. In accordance with the invention, however, the outer ceramic layer 122 prevents nickel exposure to the patient and permits use of a relatively high nickel content shaft 120 in the rod 110.

It has been discovered that the use of relatively high nickel content material (such as cobalt chromium alloy, stainless steel, etc.) in implementing the shaft 120 results in desirable strength properties of the rod 110 and, therefore permits a smaller diameter rod 110 than otherwise would be available using conventional techniques. For example, in the lumbar spine a common titanium rod diameter is in the range of 6 mm. This rod diameter is chosen at least in part because of the bending stress and bending deflection characteristics of titanium. A relatively high nickel content material, such as cobalt chromium alloy, however, exhibits much more desirable bending stress and bending deflection characteristics as compared with titanium. Thus, significantly smaller rod diameters (anywhere from about 10% to 41% smaller) may be employed using the rod 110 of the present invention. This functionality of the rod 110 will be discussed in more detail below.

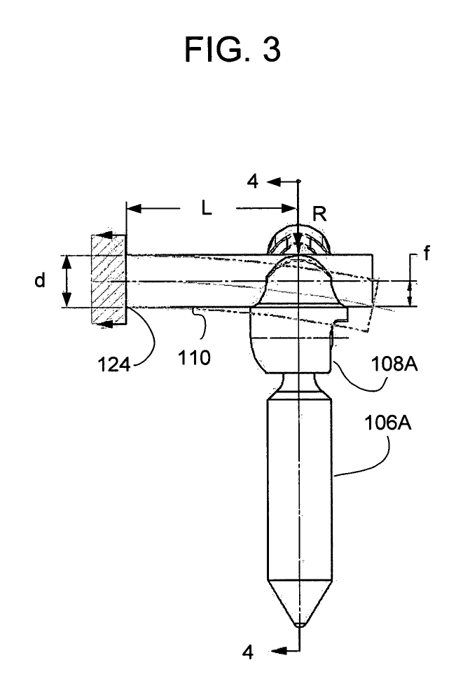

First, the relationship between an improved bending yield strength and rod diameter will be considered in terms of a theoretical cantilever beam configuration illustrated in FIG. 3. As shown, the rod 110 (of diameter d) is fixed at one end 124 as would exist when the rod 110 is fixed to one of two pedicle screws 106 in the system 100 of FIG. 1. The rod is subject to a load R in the direction of the arrow (labeled R), which load is applied a distance L (the beam length) from the end 124. This loading would also exist in response to fixing the rod 110 to a second pedicle screw 106A using an anchor 108A. The response of the rod 100 to the load L is to deflect by a distance of f. Given this elastic cantilever beam model, the yield strength criterion of the rod 110 is applied to determine a relationship with the beam (rod) diameter.

The bending stress o of a circular cross-section may be expressed using, for example, “Marks' Standard Handbook for Mechanical Engineers”, 9^{th} Edition, as follows:
σ=M×C/I [1]
where σ is the bending stress, M is the bending moment, C is the distance between the neutral axis and outer fiber, and I is the cross-sectional moment of inertia. The section modulus is I/C. It is noted that other bending stress a σ formulae of other cross-sectional geometries may also be considered, although the circular cross-section is chosen here for illustration purposes.

The distance C between the neutral axis and outer fiber may be expressed as follows:
C=d/2 [2]
And the cross-sectional moment of inertia I, may be expressed using the following formula:
I=π d⁴/64 [3]

Equations [2] and [3] may be substituted into equation [1] to obtain an expression of the bending stress as a function of the rod diameter:
σ=32M/(π d³) [4]

A ratio of the yield strengths of a base metal plus ceramic coating and an uncoated base metal may be expressed as follows:
σ_{Ti}/σ_{Co}=(d_{Co}/d_{Ti})³ [5]
or
d_{Co}=d_{Ti}(σ_{Ti}/σ_{Co})^{1/3} [6]
where d_{Co} is the diameter of the rod 110 with the ceramic coating, d_{Ti} is the diameter of the rod of base metal with no coating, σ_{Ti} is the yield strength of the rod with base metal and no coating, and σ_{Co} is the yield strength of the rod 110 with the ceramic coating.

The difference in yield strength between high nickel metals and low nickel metals approved for surgical implants can be as high as about 1.5-5 times (see, for example, ASTM standards for metal surgical implants). Therefore, according to equation [6], an increase in yield strength of about 1.5-5 times results in a rod diameter reduction of 13%-41% (0.87-0.59) without sacrificing strength of the construct. Thus, a comparison criterion may be expressed as follows:
d_{Co}=K d_{Ti}, [7]
where K is a comparison coefficient of about 0.87>K>0.59.

The criterion expression [7] describes one aspect of the reduction in rod diameter d that may be enjoyed in a system employing a rod 110 in accordance with the invention.

Next, the relationship between an improved bending displacement and rod diameter will be considered in terms of the theoretical cantilever beam configuration illustrated in FIG. 3. While the invention is not limited to any particular theory of operation, in this comparison it assumed that equal beam deflection as between a high nickel metal rods with ceramic coating and a low nickel metal rod is a design criterion.

The bending deflection f may be expressed (e.g., using “Marks' Standard Handbook for Mechanical Engineers”, 9^{th} Edition) as follows:
f=RL³/(3EI) [8]
where f is the beam (rod) deflection, and E is the modulus of elasticity (the other variables have been defined above).

Given the design criterion that the beam displacements are to be equal under equivalent bending load between beams of differing materials, then the following expression must hold:
f_{Ti}=f_{Co} [9]
where f_{Ti} is the displacement of a beam of low nickel content and f_{Co} is the beam displacement of a beam of high nickel content and coated with ceramic.

A relationship between modulus of elasticity and cross-sectional moment of inertia may be obtained by substituting equation [8] into equation [9] for equal R and L:
E_{Co}I_{Co}=EₜᵢIₜᵢ [10]
where E_{Co} is the modulus of elasticity and I_{Co} is the cross-sectional moment of inertia, respectively, of a beam of high nickel content and coated with ceramic, and E_{Ti} is the modulus of elasticity I_{Ti} is the cross-sectional moment of inertia, respectively, of beam of low nickel content.

Given that I=π d⁴/64 (equation [11]), and substituting equation [11] into equation [10], one obtains the following expression:
E_{co}d⁴_{co}=Eₜᵢd⁴ₜᵢ [12]
and
d_{Co}=d_{Ti}(E_{Ti}/E_{Co})^{1/4} [13]

Differences in modulus of elasticity between high nickel metals and low nickel metals (such as titanium alloys) approved for surgical implant manufacturing can be as high as about 1.5-3 times (see, for example, ASTM standards for metal surgical implants). Therefore, according to equation [13], an increase in the modulus of elasticity of about 1.5-3 times results in a rod diameter reduction of about 10%-24% (0.9-0.76) without sacrificing strength of the construct. Thus, a comparison criterion may be expressed as follows:
d_{Co}=N d_{Ti}, [14]
where N is a comparison coefficient of about 0.90>N>0.76. Combining formula [7] and [14], one obtains the relationship between high nickel (coated) and low nickel (uncoated) rod diameters:
d_{Co}=max(K, N)d_{Ti} [14a],
which satisfies both maximum yield and equal displacement criteria.

In general the outer layer coating 122 protects against nickel sensitivity that may be associated with uncoated high nickel content metals and metal alloys, such as cobalt chromium alloy or stainless steel. The coated cobalt chromium alloy or coated stainless steel (with high nickel content) have higher yield and fatigue strength than titanium alloy or steel (low nickel content). Further, the high nickel content rods coated with ceramic (such as titanium nitrite) have better wear resistance than low nickel content rods owing to the coating hardening the contact surfaces of the rod 110. Among the mechanical benefits of the rod 110 is a reduced rod diameter with equal bending strength and/or equal deflection (whichever criteria produces larger diameter). This results in less invasive and a less bulky system 100.

The above analysis take into consideration the bending yield strength and deflection characteristics of rods of low nickel content and high nickel content (coated with ceramic).

By way of example, a standard titanium rod (uncoated) for use in the lumbar spine typically exhibits a rod diameter in the range of about 5.30 mm to about 6.80 mm. A standard titanium rod (uncoated) for use in the thoracic spine typically exhibits a rod diameter in the range of about 3.90 mm to about 5.50 mm. A standard titanium rod (uncoated) for use in the cervical spine typically exhibits a rod diameter in the range of about 1.90 mm to about 2.80 mm.

By employing, for example, a cobalt chromium alloy, or stainless steel shaft 120 coated with a ceramic 122, such as titanium nitrite, substantially similar performance in the lumbar region of the spine may be obtained from the rod 110 with a diameter in the range of about 4.03 to 6.12 mm. A desirable diameter may be in the range of about 4.00 to 5.25 mm. In the thoracic spine a rod 110 in accordance with one or more embodiments of the invention may have a diameter in the range of about 2.96 to 4.95 mm. A desirable diameter may be in the range of about 2.90 to 3.85 mm. In the cervical spine, a rod 110 in accordance with one or more embodiments of the invention may have a diameter in the range of about 1.44 to 2.52 mm. A desirable diameter may be in the range of about 1.40 to 1.85 mm.

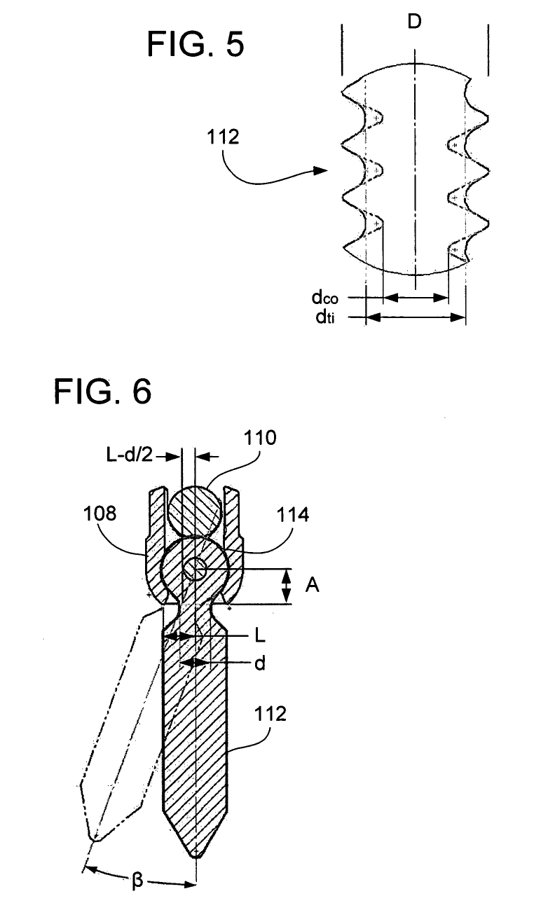

Reference is now made to FIGS. 4 and 5, which illustrate cross-sectional views of a preferred pedicle screw 106, and a screw/tulip configuration, respectively, in accordance with one or more embodiments of the present invention. The pedicle screw 106 includes a threaded screw or post 112 that is operable to engage a bore made in the bone 102, 104. The pedicle screw 106 also includes a neck 116 depending from one end of the screw or post 112 and a head 114 depending from the neck 116. The head may be sized and shaped to engage the anchor seat 108. In the particular embodiments illustrated in FIGS. 4 and 5, the head 114 and the anchor seat 108 are designed such that the anchor seat 108 may articulate with respect to the pedicle screw 106. It is understood, however, that this configuration is shown for illustration purposes only and that other structural configurations and details may be employed without departing from the scope of various embodiments of the invention.

The pedicel screw 106 is preferably formed of a first metal or metal alloy 118A and an outer layer 118B of ceramic coating the metal or metal alloy 118A. The first metal or metal alloy 118A is preferably of substantial nickel content, such as greater than about 0.3%. For example, the first metal or metal alloy 118A may be a cobalt chromium alloy, stainless steel, etc. The outer layer 118B of ceramic is of a characteristic such that exposure of nickel to the patient, or migration of the nickel into the patient, is inhibited. For example, the outer ceramic layer 118B may be titanium nitrite. The thickness of the ceramic outer layer 118B is preferably in the range of about 0.25 to 12 microns. The ceramic outer layer 118B is preferably deposited onto the first metal of metal alloy 118A using the Physical Vapor Deposition (PVD) vacuum system or any other of the known and suitable approaches for bonding materials together.

In patients with nickel sensitivity, a relatively high level of nickel in an implanted device may be intolerable. In accordance with the invention, however, the outer ceramic layer 118B prevents nickel exposure to the patient and permits use of a relatively high nickel content pedicle screw 106.

It has been discovered that the use of relatively high nickel content material (such as cobalt chromium alloy, stainless steel, etc.) in implementing the screw 106 results in desirable strength properties and, therefore permits at least one of: smaller head dimensions, smaller neck diameter, smaller minor shaft diameter of the post 112, increased thread area and bone purchase, and increased articulation as between the screw and the anchor—as compared with pedicle screws implemented with low nickel materials, such as titanium.

The mechanical dimensions of a pedicle screw are determined at least in part by the bending stress, thread purchase, and bending deflection characteristics of titanium. A relatively high nickel content material, such as cobalt chromium alloy, however, exhibits much more desirable characteristics as compared with titanium. Thus, more desirable screw dimensions may be employed using the pedicle screw 106 of the present invention. This functionality will be discussed in more detail below.

It is noted that the relationships of the rod diameter discussed above may be applied with equal weight to diameters of various parts of the screw 106, such as the neck 116 and the minor diameter of the post 112.

With reference to FIG. 5 (which is a cross sectional view of a threaded portion of the pedicle screw 106), a relationship between the minor diameter of the post 112 and the thread area (and purchase) of the screw 106 may be developed as between a high nickel content, ceramic coated screw and a low nickel content, uncoated screw. The minor diameter of the threads may be determined based on maximum yield strength criteria, while the major diameter of the threads is held fixed for both screws.

The engagement area A of a single thread may be expressed as follows:
A=n(D²−d²)/4 [21]
where A is the residual area of the single thread, D is the major (outside) diameter of the thread, and d is the minor diameter of the thread.

Assuming that D does not change between the high nickel content, ceramic coated screw and the low nickel content, uncoated screw, then the following expression may be employed:
A_{Co}−A_{Ti}=π(D²−d_{Co}²)/4−π(D²−d_{Ti}²)/4 [22]
where A_{Co} is the residual area and d_{Co} is the minor diameter of the high nickel content, ceramic coated screw, and A_{Ti} is the residual area and d_{Ti} is the minor diameter of the low nickel content, uncoated screw.

Substituting [7] into [21], yields:
A_{Co}−A_{Ti}=π(D²−(Kd_{Ti})²)/4−π(D²−d_{Ti}²)/4 [23]
and
A_{Co}−A_{Ti}=πd_{Ti}²(1−K²)/4 [24]
where 0.65>(1−K²)>0.24 [25]

By dividing both sides of formula [25] by (π d_{Ti}²/4), one can see from that use of coated high nickel material results in an increase in contact area of 24%-65% per each engaged thread, which is directly proportional to the pull-out force (purchase) associated with screw stability after implantation.

In general the outer layer coating 118B protects against nickel sensitivity that may be associated with uncoated high nickel content metals and metal alloys, such as cobalt chromium alloy or stainless steel. The coated cobalt chromium alloy or coated stainless steel (with high nickel content) have higher yield and fatigue strength than titanium alloy or steel (low nickel content). Further, the high nickel content screw 106 coated with ceramic (such as titanium nitrite) have better wear resistance than low nickel content screws owing to the coating hardening the contact surfaces of the screw 106. Among the mechanical benefits of the screw 106 are smaller head dimensions, smaller neck diameter, smaller minor shaft diameter of the post 112, increased thread area and bone purchase, smaller outside diameter and smaller minor diameter without reduced screw strength (to reduce chances of bone fracture during screw insertion), and increased articulation as between the screw and the anchor−as compared with pedicle screws implemented with low nickel materials, such as titanium.

By way of example, a standard titanium screw (uncoated) for use in the lumbar spine typically exhibits a screw minor diameter in the range of about 2.80 mm to about 3.30 mm, and a screw neck diameter in the range of about 3.88 mm to about 5.73 mm. A standard titanium screw (uncoated) for use in the thoracic spine typically exhibits a screw minor diameter in the range of about 2.97 mm to about 3.56 mm, and a screw neck diameter in the range of about 3.12 mm to about 4.35 mm. A standard titanium screw (uncoated) for use in the cervical spine typically exhibits a screw minor diameter in the range of about 1.98 mm to about 2.35 mm, and a screw neck diameter in the range of about 2.13 mm to about 2.96 mm.

By employing, for example, a cobalt chromium alloy, or stainless steel screw coated with a ceramic, such as titanium nitrite, substantially similar performance in the lumbar region of the spine may be obtained from the screw 106 with a screw minor diameter in the range of about 1.65 mm to about 2.87 mm, and a screw neck diameter in the range of about 2.29 mm to about 4.99 mm. A desirable screw minor diameter may be in the range of about 1.60 mm to about 2.75 mm, and a desirable screw neck diameter may be in the range of about 2.25 mm to about 3.85 mm. In the thoracic spine a screw 106 in accordance with one or more embodiments of the invention may a screw minor diameter in the range of about 1.75 mm to about 3.10 mm, and a screw neck diameter in the range of about 1.84 to about 3.78 mm. A desirable screw minor diameter may be in the range of about 1.70 mm to about 2.95 mm, and a desirable screw neck diameter may be in the range of about 1.80 mm to about 3.05 mm. In the cervical spine a screw 106 in accordance with one or more embodiments of the invention may a screw minor diameter in the range of about 1.17 mm to about 2.04 mm, and a screw neck diameter in the range of about 1.26 to about 2.58 mm. A desirable screw minor diameter may be in the range of about 1.10 mm to about 1.95 mm, and a desirable screw neck diameter may be in the range of about 1.20 mm to about 2.05 mm. (As will be discussed below, improved articulation may also result in each of the lumbar, thoracic and cervical regions of the spine).

With reference to FIG. 6 (which is a cross sectional view of the pedicle screw and anchor arrangement), a relationship between the maximum permissible articulation angle (between the screw and the anchor) and the neck diameter of the screw 106 may be developed as between a high nickel content, ceramic coated screw and a low nickel content, uncoated screw. The neck diameter may be determined based on the maximum yield strength criteria. Using the geometrical arrangement of FIG. 6, the following relationship concerning the angle of articulation, β, may be expressed:
tan β=(L−d/2)/A [15]
where β is the angle of articulation, L is the neck length, d is the neck diameter, and A is the distance between the screw head center and the neck contact point.

A difference in articulation angles between a high nickel content, ceramic coated screw and a low nickel content, uncoated screw may be expressed as follows:
tan β_{Co}−tan β_{Ti}=(d_{Ti}−d_{Co})/(2A) [16]
where β_{co} is the articulation angle and d_{Co} is the minor diameter, respectively, of a high nickel content, ceramic coated screw, and βₜᵢ is the articulation angle and d_{Ti} is the minor diameter, respectively, of a low nickel content, uncoated screw.

From equation [7] above a relationship between the minor diameter of the high nickel content, ceramic coated screw and the minor diameter of the low nickel content, uncoated screw may be expressed as follows:
d_{Co}=K d_{Ti}, [17]

where K is a comparison coefficient 0.87>K>0.59. Substituting equation [17] into equation [16] results in to following expression:
tan β_{Co}−tan β_{Ti}=(1−K)d_{Ti}/(2A) [18]

Thus a design coefficient DSGN relationship may be expressed as follows:
DSGN=d_{Ti}/(2A) [19]
which may stay constant for specific screw design, which results in the relationship:
0.41≧(1−K)≧0.13 [20]
which depends on the difference in yield strength between high nickel content, ceramic coated screws and low nickel content, uncoated screws.

Formula [18] demonstrates the improvement in articulation angle due to the use of coated high nickel content material screws. By dividing both sides of formula [18] by DSGN, one can see that use of coated high nickel content material for a specific screw design results in improvement of Δ tan/DSGN by 13%-41%.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A rod for interconnecting at least two pedicle screws implanted in adjacent vertebrae of a spine, comprising:
an elongate shaft formed of a metal or metal alloy of substantial nickel content; and
an outer layer of ceramic coating the shaft such that exposure of the nickel to a patient is inhibited.

2. The rod of claim 1, wherein the shaft is formed of at least one of a cobalt chromium alloy and a steel alloy.

3. The rod of claim 2, wherein the outer layer is formed of titanium nitrite.

4. The rod of claim 2, wherein the shaft is formed of stainless steel.

5. The rod of claim 1, wherein the rod is adapted for use in a lumbar region of the spine and a diameter of the shaft is between about 4.03 to 6.12 mm.

6. The rod of claim 1, wherein the rod is adapted for use in a lumbar region of the spine and a diameter of the shaft is between about 4.00 to 5.25 mm.

7. The rod of claim 1, wherein the rod is adapted for use in a thoracic region of the spine and a diameter of the shaft is between about 2.96 to 4.95 mm.

8. The rod of claim 1, wherein the rod is adapted for use in a thoracic region of the spine and a diameter of the shaft is between about 2.90 to 3.85 mm.

9. The rod of claim 1, wherein the rod is adapted for use in a cervical region of the spine and a diameter of the shaft is between about 1.44 to 2.52 mm.

10. The rod of claim 1, wherein the rod is adapted for use in a cervical region of the spine and a diameter of the shaft is between about 1.40 to 1.85 mm.

11. The rod of claim 1, wherein a concentration of nickel in the metal or metal alloy is more than about 0.3%.

12. A screw for implantation into spinal vertebrae, comprising:
a metal or metal alloy of substantial nickel content; and
an outer layer of ceramic coating the metal such that exposure of the nickel to a patient is inhibited.

13. The screw of claim 12, wherein the metal or metal alloy is formed of at least one of a cobalt chromium alloy and a steel alloy.

14. The screw of claim 13, wherein the metal or metal alloy is formed of stainless steel.

15. The screw of claim 12, wherein the outer layer is formed of titanium nitrite.

16. The screw of claim 12, further comprising:
a threaded shaft having a minor diameter and a major diameter;
a neck depending from one end of the threaded shaft; and
an at least partially spherical head coupled to the neck.

17. The screw of claim 16, wherein the screw is adapted for use in a lumbar region of the spine and at least one of:
the minor diameter is in the range of about 1.65 mm to about 2.87 mm; and
the neck diameter in the range of about 2.29 mm to about 4.99 mm.

18. The screw of claim 16, wherein the screw is adapted for use in a lumbar region of the spine and at least one of:
the minor diameter is in the range of about 1.60 mm to about 2.75 mm; and
the neck diameter in the range of about 2.25 mm to about 3.85 mm.

19. The screw of claim 16, wherein the screw is adapted for use in a thoracic region of the spine and at least one of:
the minor diameter is in the range of about 1.75 mm to about 3.10 mm; and
the neck diameter in the range of about 1.84 to about 3.78 mm.

20. The screw of claim 16, wherein the screw is adapted for use in a thoracic region of the spine and at least one of:
the minor diameter is in the range of about 1.70 mm to about 2.95 mm; and
the neck diameter in the range of about 1.80 mm to about 3.05 mm.

21. The screw of claim 16, wherein the screw is adapted for use in a cervical region of the spine and at least one of:
the minor diameter is in the range of about 1.17 mm to about 2.04 mm; and
the neck diameter in the range of about 1.26 to about 2.58 mm.

22. The screw of claim 16, wherein the screw is adapted for use in a cervical region of the spine and at least one of:
the minor diameter is in the range of 1.10 mm to about 1.95 mm; and
the neck diameter in the range of about 1.20 mm to about 2.05 mm.

23. The screw of claim 12, wherein a concentration of nickel in the metal or metal alloy is more than about 0.3%.
